(19) United States
(12) Patent Application Publication
(10) Pub. No.: US 2013/0046003 A1
(43) Pub. Date: Feb. 21, 2013

(54) PHARMACEUTICAL COMPOSITIONS COMPRISING 4-BROMO-N-(IMIDAZOLIDIN-2-YLIDENE)-1H-BENZIMIDAZOL-5-AMINE FOR TREATING RETINAL DISEASES

(71) Applicants: Mohammed I. Dibas, Laguna Niguel, CA (US); John E. Donello, Dana Point, CA (US); Daniel W. Gil, Corona Del Mar, CA (US); James A. Burke, Santa Ana, CA (US)

(72) Inventors: Mohammed I. Dibas, Laguna Niguel, CA (US); John E. Donello, Dana Point, CA (US); Daniel W. Gil, Corona Del Mar, CA (US); James A. Burke, Santa Ana, CA (US)

(21) Appl. No.: 13/555,070

(22) Filed: Jul. 20, 2012

Related U.S. Application Data
(60) Provisional application No. 61/510,536, filed on Jul. 22, 2011.
(60) Provisional application No. 61/510,743, filed on Jul. 22, 2011.

(51) Int. Cl. A61K 31/4184; A61P 9/10; A61P 25/00; A61P 27/02

(52) U.S. Cl. 514/392

(57) ABSTRACT

The present invention relates to a method for treating retinal diseases and a method for retinal neuroprotection, in a patient in need thereof which comprises of administering a therapeutically effective amount of a pharmaceutical composition comprising a therapeutically effective amount of 4-bromo-N-(imidazolidin-2-ylidene)-1H-benzimidazol-5-amine or pharmaceutically acceptable salts thereof.

### RELATED APPLICATION

This application claims the benefit of U.S. Provisional Application Ser. No. 61/510,536, filed Jul. 22, 2011, and U.S. Provisional Application Ser. No. 61/510,743, filed Jul. 22, 2011, each of which is incorporated herein by reference in its entirety.

### BACKGROUND OF THE INVENTION

1. Field of the Invention

The present invention relates to a method for treating retinal diseases and a method for retinal neuroprotection in a patient in need thereof which comprises administering a therapeutically effective amount of a pharmaceutical composition comprising a therapeutically effective amount of 4-bromo-N-(imidazolidin-2-ylidene)-1H-benzimidazol-5-amine or pharmaceutically acceptable salts thereof.

2. Description of the Prior Art

Three alpha 1 and three alpha 2 adrenergic receptors have been characterized by molecular and pharmacological methods. Activation of these alpha 2 receptors evokes physiological responses with useful therapeutic actions. Alpha-2-adrenergic receptors have been identified in the retina. A need exists for methods and compounds for use in the treatment of diseases of the retina. In human retinal diseases, blindness is often caused by injury and death of photoreceptors and ganglion cells, such as in inherited retinal degenerations and in age-related macular generation, glaucoma, and optic nerve injury.

Compound 4-bromo-N-(imidazolidin-2-ylidene)-1H-benzimidazol-5-amine is known as a potent alpha 2-adrenergic receptor pan agonist, activating all three alpha-2 receptor subtypes. 4-bromo-N-(imidazolidin-2-ylidene)-1H-benzimidazol-5-amine is disclosed U.S. Pat. No. 6,316,637 and may be prepared according to the disclosure of U.S. Pat. No. 6,495,583 B1; each patent is hereby incorporated by reference in its entirety. Acheampong et al. have shown in Xenobiotica, February 2007, Vol. 37(2), pages 205-220 that this compound was found in trace amounts in the urine of rats after administration of an oral dose of brimonidine tartrate. Brimonidine is compound (5-bromo-quinoxalin-6-yl)-imidazolidin-2-ylidene-amine and the tartrate salt is sold under the trademark ALPHAGAN®P (available from Allergan, Inc.).

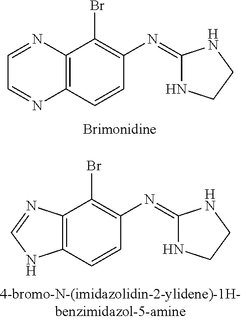 [SMILES: Brc1c(N=C2NCCN2)ccc2nccnc12 ; Brc1c(N=C2NCCN2)ccc2[nH]cnc12]

U.S. Pat. No. 6,066,675 discloses methods for the stimulation of growth factor expression and for treatment of retinal diseases with alpha- and beta-adrenergic agonists.

### SUMMARY OF THE INVENTION

The present invention provides pharmaceutical compositions, containing 4-bromo-N-(imidazolidin-2-ylidene)-1H-benzimidazol-5-amine as active ingredient for modulating the alpha 2 adrenergic receptors. We have now discovered that the pharmaceutical compositions of 4-bromo-N-(imidazolidin-2-ylidene)-1H-benzimidazol-5-amine are useful for the treatment of retinal diseases and for retinal neuroprotection in mammals, including humans.

It is an object of this invention to provide a pharmaceutical composition for treating retinal diseases in a patient suffering thereof, comprising a therapeutically effective amount of 4-bromo-N-(imidazolidin-2-ylidene)-1H-benzimidazol-5-amine or a pharmaceutically acceptable salt thereof together with a pharmaceutically acceptable carrier, diluent or excipient.

It is a further object of the invention to provide a method for treating retinal diseases in a patient suffering thereof, comprising administering to said patient a pharmaceutical composition comprising a therapeutically effective amount of 4-bromo-N-(imidazolidin-2-ylidene)-1H-benzimidazol-5-amine or a pharmaceutically acceptable salt thereof together with a pharmaceutically acceptable carrier, diluent or excipient.

It is a further of the invention to provide a pharmaceutical composition for retinal neuroprotection in a patient in need thereof, comprising a therapeutically effective amount of 4-bromo-N-(imidazolidin-2-ylidene)-1H-benzimidazol-5-amine or a pharmaceutically acceptable salt thereof together with a pharmaceutically acceptable carrier, diluent or excipient.

It is a further object of the invention to provide a method for retinal neuroprotection in a patient in need thereof, comprising administering to said patient a pharmaceutical composition comprising a therapeutically effective amount of 4-bromo-N-(imidazolidin-2-ylidene)-1H-benzimidazol-5-amine or a pharmaceutically acceptable salt thereof together with a pharmaceutically acceptable carrier, diluent or excipient.

### BRIEF DESCRIPTION OF THE DRAWINGS

FIG. 1 shows 4-bromo-N-(imidazolidin-2-ylidene)-1H-benzimidazol-5-amine reduced the damage caused by blue light.

FIG. 2 shows 4-bromo-N-(imidazolidin-2-ylidene)-1H-benzimidazol-5-amine enhanced visual acuity in normal Dutch Belted (DB) Rabbits.

FIG. 3: shows the effect of 4-bromo-N-(imidazolidin-2-ylidene)-1H-benzimidazol-5-amine protection on retinol ganglion cells (RGC) from damage induced by optic nerve crush in Sprague Dawley (SD) rats.

### DETAILED DESCRIPTION OF THE INVENTION

In one aspect of the invention, there is provided a method for treating retinal diseases in a patient suffering thereof which comprises, consists essentially of or consists of administering a therapeutically effective amount of a pharmaceutical composition comprising, consisting essentially of or consisting of a therapeutically effective amount of 4-bromo-N-(imidazolidin-2-ylidene)-1H-benzimidazol-5-amine or pharmaceutically acceptable salts thereof.

Retinal diseases which may be treated with pharmaceutical compositions containing as active ingredient 4-bromo-N-(imidazolidin-2-ylidene)-1H-benzimidazol-5-amine include, but are not limited to: macular edema, dry and wet macular degeneration, choroidal neovascularization, geographic atrophy, optic neuritis, rod dystrophies, cone-rod retinal dystrophy (CRD), diabetic retinopathy, acute macular neuroretinopathy, central serous chorioretinopathy, cystoid macular edema, diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome, retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic-surgical conditions such as sympathetic ophthalmia, uveitic retinal disease, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease, fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigement epitheliitis, proliferative vitreal retinopathy (PVR), diabetic macular edema, cytomegalovirus retinitis, glaucoma, amblyopia, stroke-induced blindness, visual system disorder in Parkinson's disease, Alzheimer's disease and multiple sclerosis, seizure-induced cortical blindness, induced visual system disorder, epileptic blindness, multiple sclerosis (MS)-induced visual system disorder, and congenital and childhood myotonic dystrophy type 1-induced visual system disorder, retinal vein-occlusive diseases, photic retinopathies, surgery-induced retinopathies (either mechanically or light-induced); toxic retinopathies; retinopathy of prematurity; viral retinopathies such as CMV or HIV retinopathy elated to AIDS; ischemic retinopathies due to venous or arterial occlusion or other vascular disorders; retinopathies due to trauma or penetrating lesions of the eye; peripheral vitro retinopathy, and inherited retinal degenerations such as, but not limited to, the various forms of retinitis pigmentosa, Usher syndrome, Refsum disease, Oguchi Disease, Malattia Leventinese, choroideremia blue cone monochromatism, Best disease, Bardet-Biedl syndrome, CMV Retinitis, Conjunctivitis (Pink Eye), Eye Herpes, Fuchs' corneal dystrophy, keratoconus, macular dystrophy, ocular hypertension, blepharitis.

In another aspect of the invention, there is provided a method for retinal neuroprotection in a patient in need thereof which comprises, consists essentially of or consists of administering a therapeutically effective amount of a pharmaceutical composition comprising, consisting essentially of or consisting of a therapeutically effective amount of 4-bromo-N-(imidazolidin-2-ylidene)-1H-benzimidazol-5-amine or pharmaceutically acceptable salts thereof.

Conditions related to retinal neuroprotection, include but are not limited to atrophy associated with dry age related macular degeneration, atrophy associated with wet age related macular degeneration, ocular hypertension, ischemia secondary to glaucoma, photoreceptor cell damage associated with retinitis pigmentosa, geographic atrophy, Stargardt's disease, acute macular neuroretinopathy, optic neuritis, diabetic retinopathy, diabetic macular edema and retinal vein occlusion.

The term “pharmaceutical composition,” as used here, means a composition that is suitable for administering to human patients for the treatment of disease.

Administration of the presently useful compound for use in the methods of this invention can include, but are not limited to, topical, oral, parenteral, intravenous, subcutaneous and other modes of systemic administration.

The compound of the invention can be administered in a therapeutically effective amount either alone or in combination with a suitable pharmaceutically acceptable carriers or excipients. Depending on the intended mode of administration, the presently useful compound may be incorporated in any pharmaceutically acceptable dosage form, such as for example, tablets, suppositories, pills, capsules, powders, liquids, solutions, infusions, suspensions, emulsions, aerosols or the like, preferably dosage forms suitable for single administration of precise dosages, or sustained release dosage forms for continuous controlled administration. Preferably, the dosage form will include an ophthalmically pharmaceutically acceptable excipient for topical application and the present compound, it may also contain other medicinal agents, pharmaceutical agents, carriers, adjutants, etc.

The compound of the invention can delivered to the eye through a variety of routes, including but not limited to intraocular, by topical application to the eye or by intraocular injection into, for example, the vitreous or subretinal (interphotoreceptor space); locally by insertion or injection into the tissue surrounding the eye; systemically through an oral route or by subcutaneous, intravenous or intramuscular injection; or via catheter or implant. The compound of the invention can be administered prior to the onset of the condition, to prevent its occurrence, such as during eye surgery, immediately after the onset of the pathological condition, or during the occurrence of an acute or protracted condition.

Liquid pharmaceutically administrable dosage forms can, for example, comprise a solution or suspension of one or more of the present compound and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980, incorporated by reference herein. The composition of the formulation to be administered contains a quantity of the presently useful compound in an amount effective to provide the desired therapeutic effect.

The amount of the presently useful compound administered is, of course, dependent on the therapeutic effect or effects desired, on the specific mammal being treated, on the severity and nature of the mammal's condition, on the manner of administration, on the potency and pharmacodynamics of the particular compound employed, and on the judgement of the prescribing physician. Generally, the therapeutically effective dosage of the presently useful compound for a topical formulation is preferably in a range of about 0.001%-5.0% up to three times daily. For systemic delivery the range would be of about 0.01 to about 100 mg/kg/day. In some embodiments, the composition includes a therapeutically effective dosage of the compound at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w).

“Pharmaceutically acceptable salt” refers to those salts or complexes which retain the biological effectiveness and properties of the free base and retain the desired biological activity of 4-bromo-N-(imidazolidin-2-ylidene)-1H-benzimidazol-5-amine and exhibit minimal or no undesired toxicological effects. The “pharmaceutically acceptable salts” according to the invention include therapeutically active, non-toxic acid salt forms, which 4-bromo-N-(imidazolidin-2-ylidene)-1H-benzimidazol-5-amine is able to form. The acid addition salt form of 4-bromo-N-(imidazolidin-2-ylidene)-1H-benzimidazol-5-amine can be obtained by treating the free base with an appropriate acid such as an inorganic acid for example: hydrochloric acid, hydrobromic acid, sulfuric acid, nitric acid, phosphoric acid and the like; or an organic acid for example: acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, p-toluenesulfonic acid, salicylic acid benzenesulfonic acid, formic and the like (Handbook of Pharmaceutical Salts, P. Heinrich Stahal & Camille G. Wermuth (Eds), Verlag Helvetica Chemica Acta-Zurich, 2002, 329-345).

4-bromo-N-(imidazolidin-2-ylidene)-1H-benzimidazol-5-amine may be formulated with efficacy enhancing components as disclosed in U.S. Pat. No. 7,491,383 B2, which is hereby incorporated by reference in its entirety.

4-bromo-N-(imidazolidin-2-ylidene)-1H-benzimidazol-5-amine is advantageous for retinal neuroprotection, including but not limited to atrophy associated with dry age related macular degeneration, atrophy associated wet age related macular degeneration, dry age related macular degeneration, ischemia secondary to glaucoma, photoreceptor cell damage associated with retinitis pigmentosa, geographic atrophy, Stargardt's disease, acute macular neuroretinopathy, optic neuritis, diabetic retinopathy, diabetic macular edema and retinal vein occlusion. Our compound of interest is also useful for enhancing vision in patients with vision loss from conditions including ocular hypertension, glaucoma and neuritis secondary to multiple sclerosis.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

### Example 1

### The Blue Light Model

In order to demonstrate the use of 4-bromo-N-(imidazolidin-2-ylidene)-1H-benzimidazol-5-amine for retinal disease treatment, use of 4-bromo-N-(imidazolidin-2-ylidene)-1H-benzimidazol-5-amine was compared to brimonidine and vehicle in the blue light model of retinal degeneration in rats. Drugs were administered via intraperitoneal injection (IP) at a dose of 1 mg/kg one hour before blue light exposure.

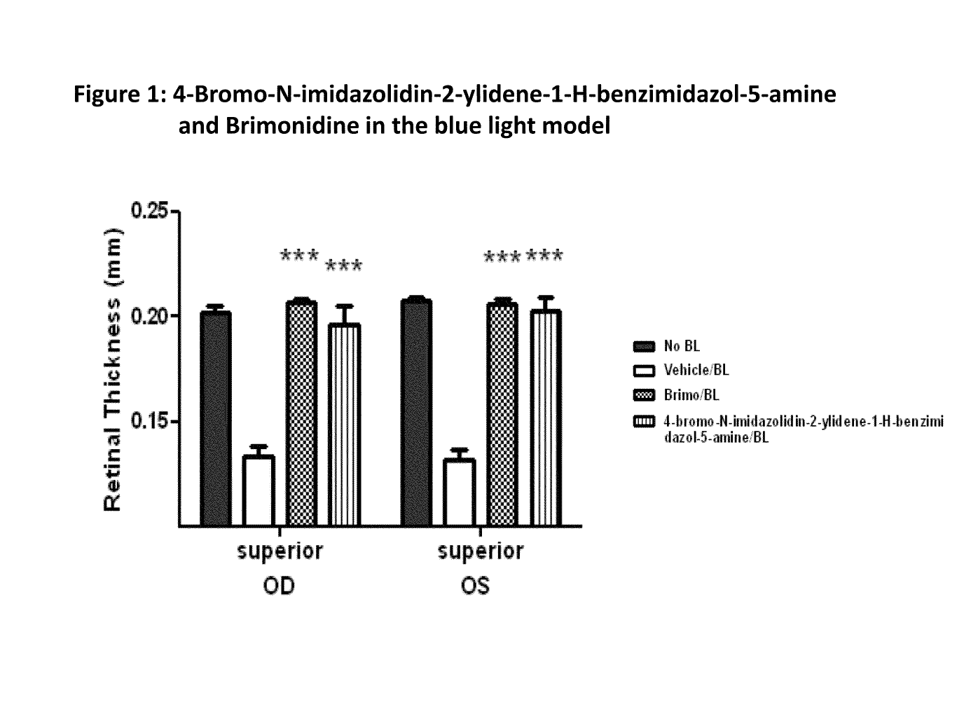

Twenty 4-month old male Sprague-Dawley rats (body weight 470-550 g) were used in this study. The animals were exposed to room light on a 12 hour lighV12 hour dark cycle before the experiment. All animals were dark adapted overnight (16-20 hours) before blue light exposure. Under the intensity of 6100-6500 lux, rats were exposed to blue light for 4 hours. After the blue light, rats were placed in the dark for another 3 days before returning to normal 12 hour light/12 hour dark. Ocular Coherence Tomography (OCT) measurement was performed at 7 days post blue light exposure. The results reported in FIG. 1 demonstrate that blue light exposure with just saline treatment leads to dramatic reduction of retinal thickness measured by OCT, particularly in the superior retina. Histology studies have shown that the reduction in retinal thickness is attributable to loss of photoreceptors. Both 4-bromo-N-(imidazolidin-2-ylidene)-1H-benzimidazol-5-amine and brimonidine treatment significantly reduced and protected the retina from the damage caused by blue light.

### Example 2

### Visual Enhancement Model

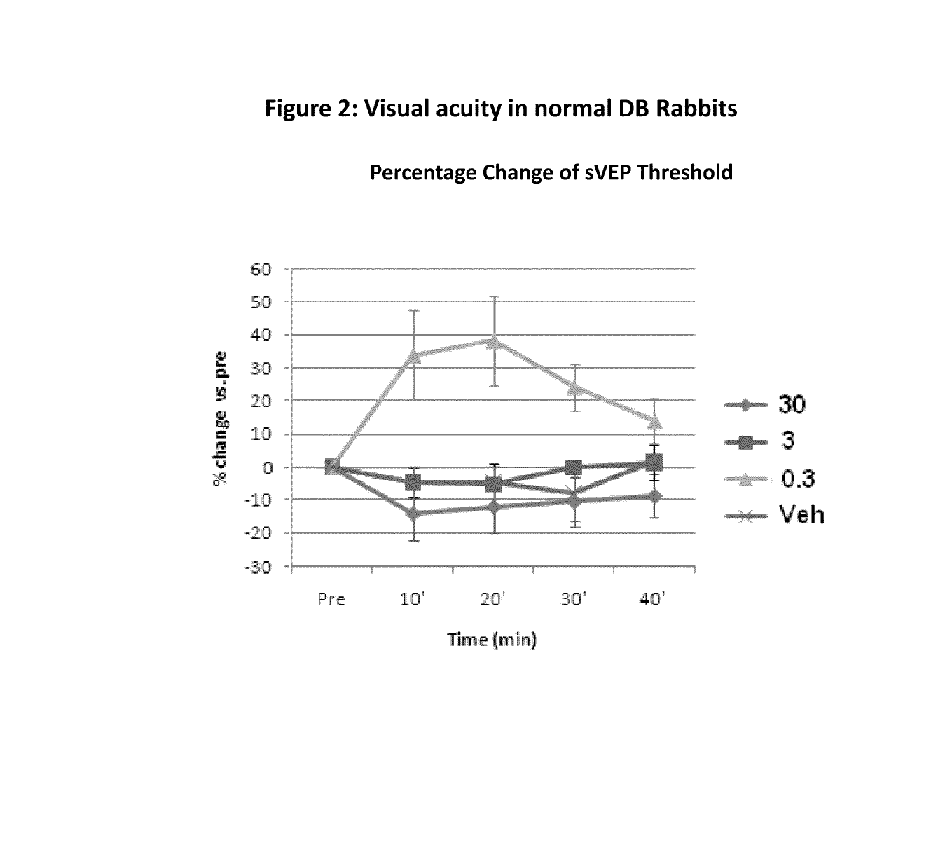

Sixteen pigmented (Dutch-Belted) rabbits weighing 2-3 kg were used to evaluate the neuroenhancement effect of 4-bromo-N-(imidazolidin-2-ylidene)-1H-benzimidazol-5-amine. Rabbits were dosed with 4-bromo-N-(imidazolidin-2-ylidene)-1H-benzimidazol-5-amine through intravenous route. Spatial sweep visual evoked potential (sVEP) acuity was assessed with PowerDiva software version 1.8. Recordings were made bilaterally in conscious animals. The results reported in FIG. 2, demonstrated that 4-bromo-N-(imidazolidin-2-ylidene)-1H-benzimidazol-5-amine enhanced visual acuity at 10-30 minutes post-dose in normal DB rabbits.

### Example 3

### The Nerve Crush Model

This example described the neuroprotective effect of 4-bromo-N-(imidazolidin-2-ylidene)-1H-benzimidazol-5-amine level in the rat nerve crush model. Sprague Dawley rats weighing 300-350 g were anesthetized with a mixture of ketamine (50 mg/kg) and xylazine (0.5 mg/kg). Lateral canthotomy was performed in the right eye and an incision was made in the superior conjunctiva adjacent to the rectus muscle. This was followed by a blunt dissection until optic nerve was exposed. A partial crush was applied to the optic nerve for 30 seconds, 2 to 3 mm distal from the globe, using calibrated cross-acting forceps. Care was taken not to interfere with retinal blood supply. 4-bromo-N-(imidazolidin-2-ylidene)-1H-benzimidazol-5-amine was administered at 0.03, 0.1, 0.3, 1 mg/kg subcutaneous injection (SC) two hours before nerve injury, the vehicle phosphate-buffered saline (PBS) was administered SC as a negative control whereas brimonidine 0.1 mg/kg was given by IP injection as a positive control. Control animals received phosphate-buffered saline (PBS) vehicle. The experiment was terminated 12-15 days post nerve crush.

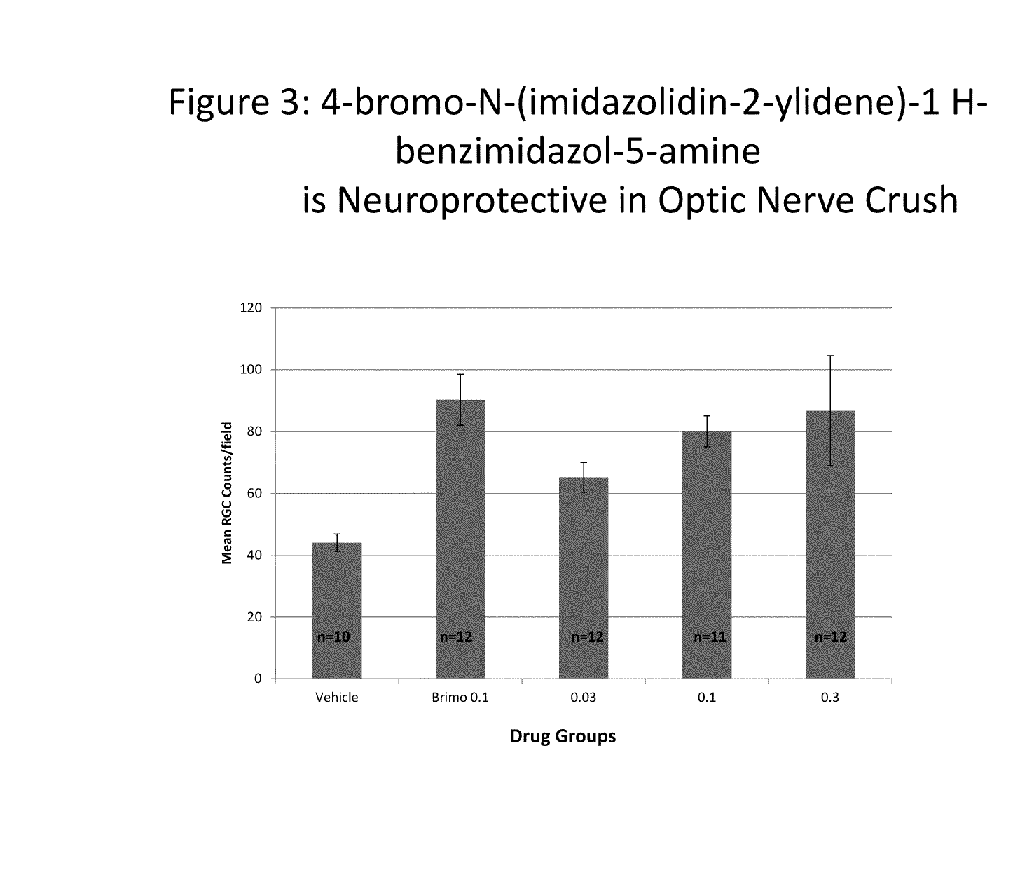

FIG. 3: shows the effect of 4-bromo-N-(imidazolidin-2-ylidene)-1H-benzimidazol-5-amine on retinol ganglion cells (RGC) decrease induced by optic nerve crush in SD rats.

### Example 4

### The Chronic Ocular Hypertension Model

Intraocular Pressure (IOP) was elevated in male Witar rats weighing 350-450 g using laser photocoagulation with blue-green argon laser (Coherent, Palo Alto, Calif.). Rats were anesthetized with a mixture of ketamine (15 mg/kg), acepromazine (1.5 mg/kg), and xylazine (0.3 mg/kg). Laser treatment was done in two parts (1-week interval) on limbal and epsiscleral veins. The amount of energy used was 1 W for 0.2 seconds, delivering a total of 150 spots (50-100 μM). Intraocular pressure was measured using tonometer (TONO-PEN: mentor, Norwell, Mass.). Rats were sedated with 3.0 mg/kg IM acepromazine during IOP measurements. Proparacaine 0.5% was applied topically on the eyes to anesthetize the cornea. Initial IOP measurements were done before laser treatment to determine baseline IOP and subsequent measurements were done once a week.

4-bromo-N-(imidazolidin-2-ylidene)-1H-benzimidazol-5-amine was administered constantly using an osmotic pump (Alzet Osmotic Pumps, Duret Corp., Cupertino, Calif.) which was inserted subcutaneously on the back at 0.03, 0.1, 0.3, 1 mg/kg per day, SC two hours before nerve injury, the vehicle PBS was administered SC as a negative control whereas brimonidine 0.1 mg/kg was given by IP injection as a positive control. Control animals received phosphate-buffered saline (PBS) vehicle. The experiment was terminated 12-15 days later. In chronic hypertension model, there is a gradual decrease in retinal ganglion cells (RGC) over a period of weeks (i.e., 30% RGC loss per week).

What is claimed is:

1. A pharmaceutical composition for treating retinal diseases in a patient suffering thereof, comprising a therapeutically effective amount of 4-bromo-N-(imidazolidin-2-ylidene)-1H-benzimidazol-5-amine or a pharmaceutically acceptable salt thereof together with one or more pharmaceutically acceptable carriers.

2. A method for treating retinal diseases in a patient suffering thereof, comprising administering to said patient a pharmaceutical composition according to claim 1.

3. The method according to claim 2, wherein the pharmaceutical composition is administered to the eye.

4. The method according to claim 2, wherein the retinal disease is selected from: age related macular degeneration, wet macular degeneration, dry macular degeneration, geographic atrophy, diabetic retinopathy, diabetic macular edema, retinitis pigmentosa, retinal vein occlusion, neuritis secondary to multiple sclerosis, Stargardt's disease, acute macular neuroretinopathy, optic neuritis and diabetic retinopathy.

5. An article of manufacture comprising packaging material and a pharmaceutical agent contained within said packaging material, wherein said pharmaceutical agent is effective for the treatment of a patient suffering from retinal diseases and wherein the packaging material comprises a label which indicates that said pharmaceutical agent can be used for treating retinal diseases and wherein said pharmaceutical agent is 4-bromo-N-(imidazolidin-2-ylidene)-1H-benzimidazol-5-amine or a pharmaceutically acceptable salt thereof.

6. A pharmaceutical composition for retinal neuroprotection in a patient in need thereof, comprising a therapeutically effective amount of 4-bromo-N-(imidazolidin-2-ylidene)-1H-benzimidazol-5-amine or a pharmaceutically acceptable salt thereof together with one or more pharmaceutically acceptable carrier.

7. A method for retinal neuroprotection in a patient in need thereof, comprising administering to said patient a pharmaceutical composition according to claim 6.

8. The method according to claim 7, wherein the pharmaceutical composition is administered to the eye.

9. The method according to claim 7, wherein the patient suffers from: atrophy associated with dry age related macular degeneration, wet age related macular degeneration, atrophy associated with wet age related macular degeneration, ocular hypertension, ischemia secondary to glaucoma, photoreceptor cell damage associated with retinitis pigmentosa, geographic atrophy, Stargardt's disease, acute macular neuroretinopathy, optic neuritis, diabetic retinopathy, diabetic macular edema and retinal vein occlusion.

10. An article of manufacture comprising packaging material and a pharmaceutical agent contained within said packaging material, wherein said pharmaceutical agent is effective for the treatment of a patient in need of retinal neuroprotection and wherein the packaging material comprises a label which indicates that said pharmaceutical agent can be used for retinal neuroprotection and wherein said pharmaceutical agent is 4-bromo-N-(imidazolidin-2-ylidene)-1H-benzimidazol-5-amine or a pharmaceutically acceptable salt thereof.
